(19) United States
(12) Patent Application Publication
(10) Pub. No.: US 2016/0081829 A1
(43) Pub. Date: Mar. 24, 2016

(54) AORTIC INSUFFICIENCY REPAIR DEVICE AND METHOD

(71) Applicant: Edwards Lifesciences Corporation, Irvine, CA (US)

(72) Inventor: Stanton J. Rowe, Newport Coast, CA (US)

(21) Appl. No.: 14/861,140

(22) Filed: Sep. 22, 2015

Related U.S. Application Data
(60) Provisional application No. 62/053,581, filed on Sep. 22, 2014.

(51) Int. Cl. A61F 2/954; A61F 2/90; A61F 2/844; A61F 2/07; A61F 2/24

(52) U.S. Cl. CPC A61F 2/954; A61F 2/07; A61F 2/2418; A61F 2/2466; A61F 2/2442; A61F 2/844; A61F 2/90; A61F 2002/072; A61F 2002/065

(57) ABSTRACT

The present application concerns embodiments of methods, systems, and apparatus for treating aortic insufficiency. Disclosed methods, systems and apparatus can also be used to treat aortic root dilation. Certain embodiments include a percutaneous or minimally invasively implantable prosthetic device, such as a stented graft, that is configured to be implanted in the sinus of Valsalva (the aortic sinuses) and anchored within one or both of the coronary arteries. An expandable prosthetic heart valve can then be implanted in the previously implanted prosthetic device. In patients suffering from root dilation, another percutaneous or minimally invasively implantable graft can be implanted within the ascending aorta.

### CROSS-REFERENCE TO RELATED APPLICATION

The present application claims the benefit of U.S. Provisional Application No. 62/053,581, filed Sep. 22, 2014.

### FIELD

This application relates to methods and apparatus for implanting prosthetic devices, and in particular, implanting prosthetic devices for treating aortic insufficiency.

### BACKGROUND

Prosthetic heart valves have been used for many years to treat cardiac valvular disorders. The native heart valves (such as the aortic, pulmonary, tricuspid and mitral valves) serve critical functions in assuring the forward flow of an adequate supply of blood through the cardiovascular system. These heart valves can be rendered less effective by congenital, inflammatory, or infectious conditions. Such conditions can eventually lead to serious cardiovascular compromise or death. For many years the definitive treatment for such disorders was the surgical repair or replacement of the valve during open heart surgery.

More recently, a transvascular technique has been developed for introducing and implanting a prosthetic heart valve using a flexible catheter in a manner that is less invasive than open heart surgery. In this technique, a prosthetic valve is mounted in a crimped state on the end portion of a flexible catheter and advanced through a blood vessel of the patient until the valve reaches the implantation site. The valve at the catheter tip is then expanded to its functional size at the site of the defective native valve, such as by inflating a balloon on which the valve is mounted. Alternatively, the valve can have a resilient, self-expanding stent or frame that expands the valve to its functional size when it is advanced from a delivery sheath at the distal end of the catheter.

Balloon-expandable valves are commonly used for treating heart valve stenosis, a condition in which the leaflets of a valve (e.g., an aortic valve) become hardened with calcium. The hardened leaflets provide a good support structure on which the valve can be anchored within the valve annulus. Further, the catheter balloon can apply sufficient expanding force to anchor the frame of the prosthetic valve to the surrounding calcified tissue. There are several heart conditions, however, that do not involve hardened valve leaflets but that are still desirably treated by valve replacement. For example, aortic insufficiency (or aortic regurgitation) occurs when an aortic valve does not close properly, allowing blood to flow back into the left ventricle. One cause for aortic insufficiency is a dilated aortic annulus, which prevents the aortic valve from closing tightly. In such cases, the leaflets are usually too soft to provide sufficient support for a balloon-expandable prosthetic valve. Additionally, the diameter of the aortic annulus may continue to vary over time, making it dangerous to install a prosthetic valve that is not reliably secured in the valve annulus. Mitral insufficiency (or mitral regurgitation) involves these same conditions but affects the mitral valve.

In addition to the dilation of the aortic annulus, in some cases aortic insufficiency is associated with dilation of the aortic root and/or the ascending aorta, which can lead to aneurisms. About 30 percent of patients suffering from aortic insufficiency require aortic root replacement, which is a difficult operation with high morbidity and mortality.

Self-expanding prosthetic valves are sometimes used for replacing defective native valves with non-calcified leaflets. Self-expanding prosthetic valves, however, suffer from a number of significant drawbacks. For example, once a self-expanding prosthetic valve is placed within the patient's defective heart valve (e.g., the aorta or mitral valve), it continues to exert an outward force on the valve annulus. This continuous outward pressure can cause the valve annulus to dilate further, exacerbating the condition the valve was intended to treat.

Accordingly, there exists a need for improved methods, systems, and apparatus for treating patients suffering from aortic insufficiency.

### SUMMARY

In one representative embodiment, a method comprises introducing a guidewire into a patient's body, advancing the guidewire until a distal end portion of the guidewire extends into the aortic root and into one of the coronary arteries, advancing a prosthetic device along the guidewire into the aortic root, aligning a side opening of the prosthetic device with the coronary artery into which the guidewire extends, and radially expanding the prosthetic device within the aortic root. The prosthetic device can be a stented graft that comprises an expandable metal frame and a blood-impermeable liner or sleeve supported on the inner and/or outer surfaces of the metal frame. The method can further comprise implanting a prosthetic valve within the prosthetic device. In certain embodiments, the prosthetic valve can have a plastically-expandable frame and can be expanded/deployed within the prosthetic device using an inflatable balloon of a delivery apparatus or an equivalent expansion mechanism. The method can further comprise implanting a stented graft in the ascending aorta of the patient to treat an aneurism or a dilated section of the ascending aorta.

In particular embodiments, two guidewires can be inserted, one into each coronary artery, and the prosthetic device can have two side openings. The prosthetic device can be advanced over the guidewires, which assist in aligning the side openings with the coronary arteries.

In another representative embodiment, an implantable prosthetic device is configured for implantation in the aortic root of a patient. The prosthetic device comprises an annular body configured to be radially compressed to a delivery state for insertion into the patient and expandable to an expanded state against the inner wall of the aortic root. The annular body has first and second openings that are configured to allow blood to flow outwardly through the openings and into the coronary arteries when the annular body is in an expanded state engaging the inner wall of the aortic root. The prosthetic device can serve as a scaffolding or anchor to receive a separate expandable prosthetic valve that is implanted within the prosthetic device.

In another representative embodiment, a medical device assembly comprises first and second guidewires, an elongated delivery apparatus having a distal end portion, and an implantable prosthetic device configured to be implanted within the aortic root of a patient's body. The prosthetic device is mounted in a radially compressed state on the distal end portion of the delivery apparatus. The prosthetic device comprises an annular body and first and second openings in the annular body, and is configured to allow blood to flow outwardly through the openings and into the coronary arteries when the annular body is in an expanded state engaging the inner wall of the aortic root. The first and second guidewires extend into and through the first and second openings, respectively, and through the annular body.

The foregoing and other objects, features, and advantages of the invention will become more apparent from the following detailed description, which proceeds with reference to the accompanying figures.

### BRIEF DESCRIPTION OF THE DRAWINGS

FIG. 1 shows the placement of guidewires in the aortic root and coronary arteries of a patient's body.

FIG. 2 shows an exemplary embodiment of an implantable prosthetic device being delivered to the aortic root along the guidewires.

FIG. 3 shows the prosthetic device implanted within the aortic root.

FIG. 4 is a front elevation view of the prosthetic device of FIGS. 2 and 3.

FIG. 5 is a top plan view of the prosthetic device of FIG. 4.

FIG. 6 shows an exemplary embodiment of an aortic graft being delivered to the ascending aorta.

FIG. 7 shows the aortic graft implanted within the ascending aorta.

FIG. 8 shows an exemplary embodiment of a prosthetic valve being delivered to the prosthetic device previously implanted within the aortic root.

FIG. 9 shows the deployment of the prosthetic valve.

FIG. 10 shows the prosthetic valve implanted within the prosthetic device.

FIG. 11 is a side elevation view of another embodiment of an implantable prosthetic device that is implantable within the aortic root of a patient.

FIG. 12 is a front elevation view of another embodiment of an implantable prosthetic device that is implantable within the aortic root of a patient.

FIG. 13 shows the prosthetic device of FIG. 12 being implanted within the aortic root of a patient.

FIG. 14 is a front elevation view of another embodiment of an implantable prosthetic device that is implantable within the aortic root of a patient.

FIG. 15 shows a prosthetic assembly implanted in the aortic valve and the aorta, according to another embodiment.

FIG. 16 is a perspective view of the sinus graft of the assembly shown in FIG. 15.

FIG. 17 is a front elevation view of the aortic stent graft of the assembly shown in FIG. 15.

### DETAILED DESCRIPTION

Disclosed below are representative embodiments of methods, systems, and apparatus used to replace deficient native heart valves with prosthetic heart valves. Embodiments of the disclosed methods, systems, and apparatus can be used, for example, to replace an aortic valve suffering from aortic insufficiency. Disclosed methods, systems and apparatus can also be used to treat aortic root dilation. Certain embodiments include a percutaneous or minimally invasively implantable prosthetic device, such as a stented graft, that is configured to be implanted in the sinus of Valsalva (the aortic sinuses) and to be anchored within one or both of the coronary arteries. An expandable prosthetic heart valve can then be implanted in the previously implanted prosthetic device. In patients suffering from root dilation, another percutaneous or minimally invasively implantable graft can be implanted within the ascending aorta.

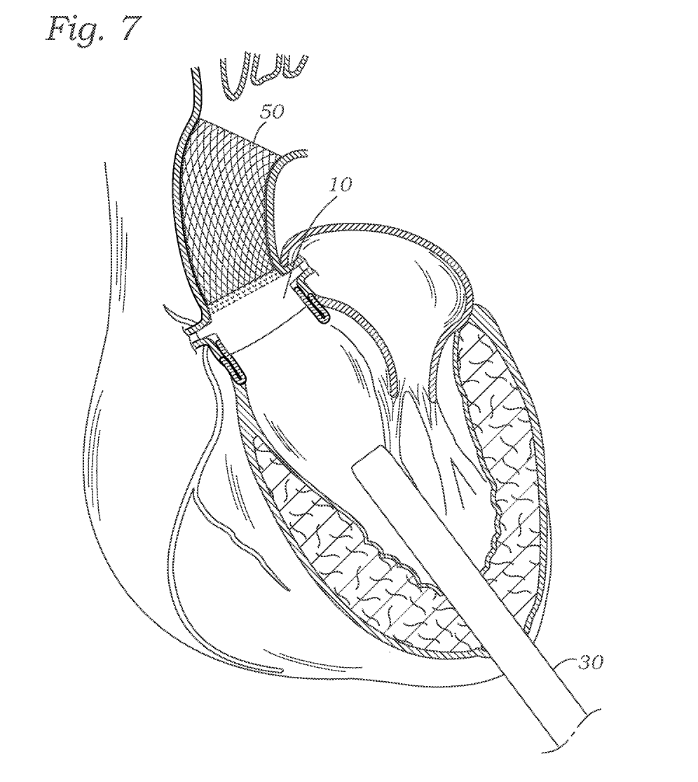

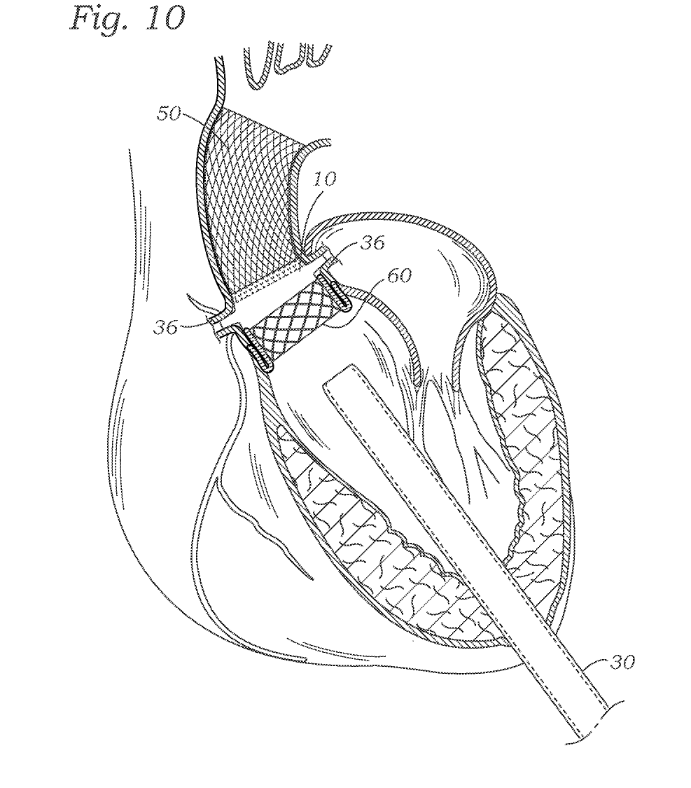

A prosthetic assembly or kit for treating aortic insufficiency and aortic root dilation can include a first stent graft 10 (FIG. 4) for implantation the sinus of Valsalva, a second graft 50 (FIG. 7) for implantation in the ascending aorta, and a prosthetic valve 60 (FIG. 10). Methods and devices for implanting these components are described in detail below.

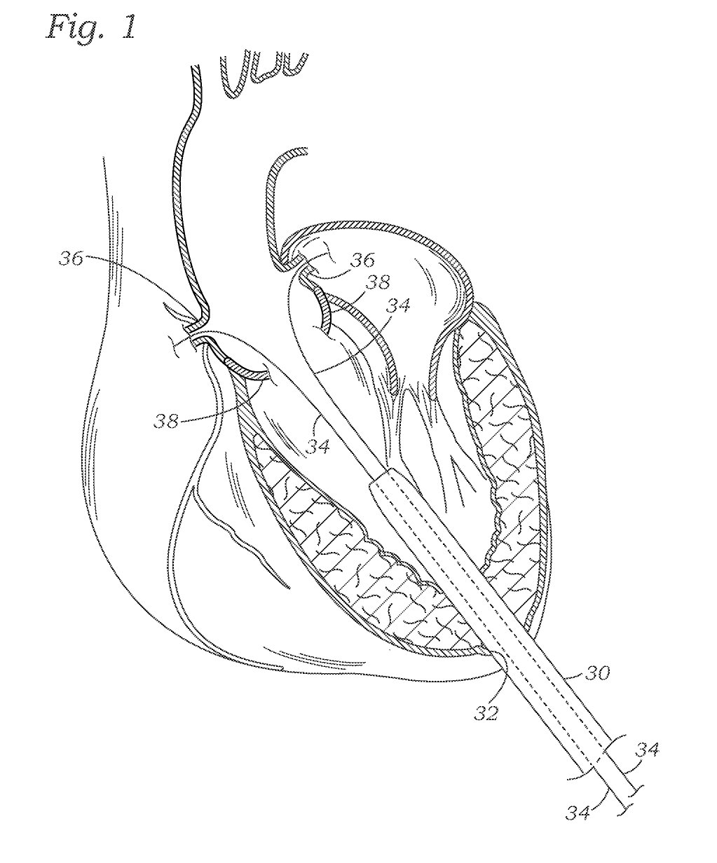

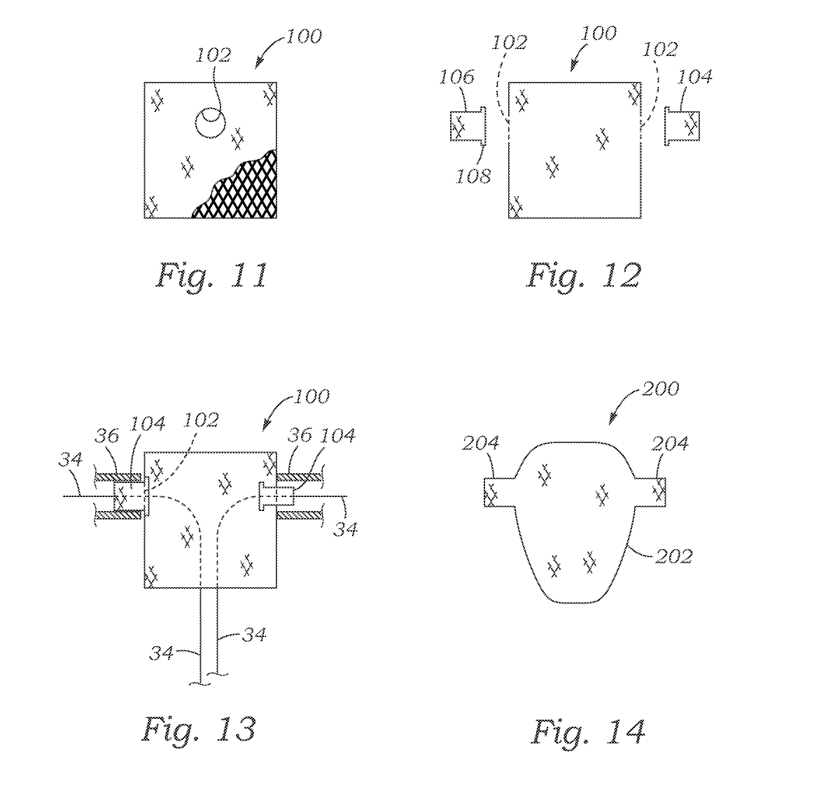

FIGS. 1-3 illustrate a method of implanting a prosthetic device, such as in the form of the stented graft 10, according to one embodiment. The graft 10 is shown in greater detail in FIGS. 4 and 5. The graft 10 in the illustrated embodiment comprises an annular main body 12 and one or two side branches or branch conduits 14 extending laterally from the main body. The main body has first and second openings 22, from which the side branches 14 extend. The main body 14 is configured to be implanted within the sinus of Valsalva while the side branches 14 are configured to extend into the coronary arteries 36, thereby assisting in anchoring the graft 10 in place against the flow of blood, as further described below. Accordingly, the graft 10 can be referred be referred to as a “sinus graft.” The main body 12 in the illustrated embodiment is cylindrical in shape, although the main body can have any of various shapes. For example, in alternative embodiments, the main body 12 can have a bulbous shape generally corresponding to the shape of the sinus of Valsalva (such as shown in FIG. 14). Such a bulbous-shaped main body can have a central portion that has a diameter that is larger than the diameters of the inflow and outflow ends of the main body.

The graft 10 in the illustrated embodiment further comprises a stent or frame 16 that supports a blood-impermeable cover, liner, or sleeve 18 extending over and covering the outside of the frame 16. In FIG. 4, a portion of the cover 18 is broken away for purposes of illustration to reveal a portion of the frame 16 underneath. The frame 16 can be made, for example, of a wire mesh or a laser cut tube, and can be radially collapsible and expandable between a radially expanded state and a radially compressed state to enable delivery and implantation within the aortic root. The wire mesh can include metal wires or struts arranged in a lattice pattern, such as the sawtooth or zig-zag pattern shown in FIG. 4, for example, but other patterns may also be used. The frame 16 can comprise a shape-memory material, such as a nickel-titanium alloy (known as “nitinol”) for example, to enable self-expansion from the radially compressed state to the expanded state. In alternative embodiments, the frame 16 can be plastically expandable from a radially compressed state to an expanded state by an expansion device, such as an inflatable balloon (not shown) for example. Such plastically expanding frames can comprise stainless steel, chromium alloys, and/or other suitable materials.

The cover 18 can comprise synthetic materials, such as polyester material or a biocompatible polymer. One example of a polyester material is polyethylene terephthalate (PET; for example, DACRON® PET (Invista, Wilmington, Del.)). Alternative materials can be used. For example, the cover 18 can comprise biological matter, such as pericardial tissue (e.g., bovine, porcine, or equine pericardium) or other biological tissue. Also, in alternative embodiments, the cover 18 can be mounted on the inside of the frame 12, rather than on the outside as is depicted in FIG. 4. In another embodiment, the prosthetic device can be provided without a cover 18 on the outside or inside of the frame and therefore comprises a bare stent or frame.

Each of the branch conduits 14 can comprise an expandable annular stent that is covered by the material forming the cover 18.

In alternative embodiments, the graft 14 can have only one opening 22 and one branch conduit 14, which are aligned within one of the coronary arteries when implanted. To avoid blocking the other coronary artery, the main body 12 can be shaped such that it does not extend over and block the coronary artery, such as by including a cut-out or recessed portion along the outflow edge of the main body 12.

As shown in FIG. 5, the graft 10 can include a valvular structure, such as one or more prosthetic leaflets 20, to permit the flow of blood through the graft in one direction. As such, the graft 10 is actually a prosthetic valve. In particular embodiments, the leaflets 20 serve as a temporary valve to regulate the flow of blood until a more robust prosthetic valve is deployed within the graft 10, as further described below. As such, the leaflets 20 can be made relatively thinner than the leaflets of the prosthetic valve to be implanted within the graft 10. The leaflets 20 can be made of synthetic materials, such as polyurethane, or biological matter, such as pericardial tissue (e.g., bovine, porcine, or equine pericardium). In certain embodiments, the leaflets 20 are capable of functioning for at least 48 hours following implantation until a more robust prosthetic valve is subsequently implanted. In alternative embodiments, the graft 10 is not provided with any prosthetic leaflets and therefore does not assist in regulating the flow of blood but still serves as anchor for a subsequently implanted prosthetic valve.

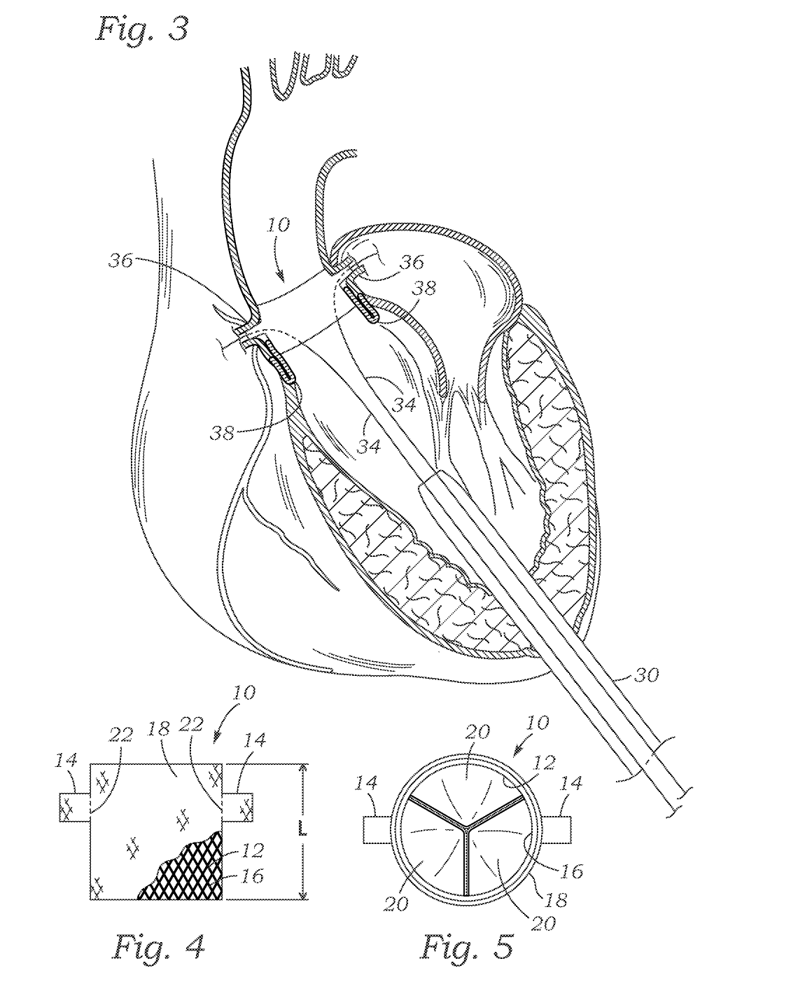

FIG. 3 shows the graft 10 implanted within the aortic root with the side branches 14 extending into the coronary arteries 36. It is important that the graft not obstruct the flow of blood into the coronary arteries. Thus, to avoid obstructing the coronary arteries, the leaflets 20 can be mounted within the main body 12 below the side branches 14 such that blood can flow through the leaflets 20 and then into the coronary arteries 36. Alternatively, the leaflets 20 can be mounted within the main body 12 above the side branches 14 such that blood from the left ventricle can still flow into the coronary arteries 36 downstream of the native leaflets 38.

In particular embodiments, the graft 10 has an overall length L of about 30 mm to about 50 mm, with about 40 mm being a specific example. When implanted in the aortic root, the outflow end portion of the graft 10 can extend a small distance into the ascending aorta, such as about 10-20 mm into the ascending aorta.

In certain embodiments, the cover 18 can extend beyond the inflow and/or outflow ends of the frame 16. Depending on the particular anatomy of the patient, the surgeon can trim the inflow and/or outflow ends of the cover 18 to achieve a desired fit within aortic root. Imaging techniques (e.g., CT scanning, ultrasound, etc.) can be used to obtain an image and measure aspects of the aortic root so that the cover 18 can be trimmed or cut to achieve a desired fit within the aortic root.

As noted above, FIGS. 1-3 illustrate a method of implanting the graft 10. In the illustrated embodiment, the graft is delivered to the implantation site in a trans-ventricular procedure via a surgical incision made in the wall of the left ventricle, for example, a transapical procedure. Desirably, a surgical incision is made at the bare spot on the lower anterior ventricle wall to provide access for insertion of medical instruments into the heart. As shown in FIG. 1, an introducer sheath 30 can be inserted through a surgical incision 32 in the left ventricle. Guidewires 34 can be inserted through the introducer sheath 30 and the native aortic valve until the distal end of each guidewire 34 is positioned within a respective coronary artery 36. The proximal ends of the guidewires (not shown) desirably extend proximally past the proximal housing of the introducer (not shown) outside of the patient's body. In the drawings, the native leaflets 38 of the aortic valve are broken away at their inner ends to better illustrate the procedure.

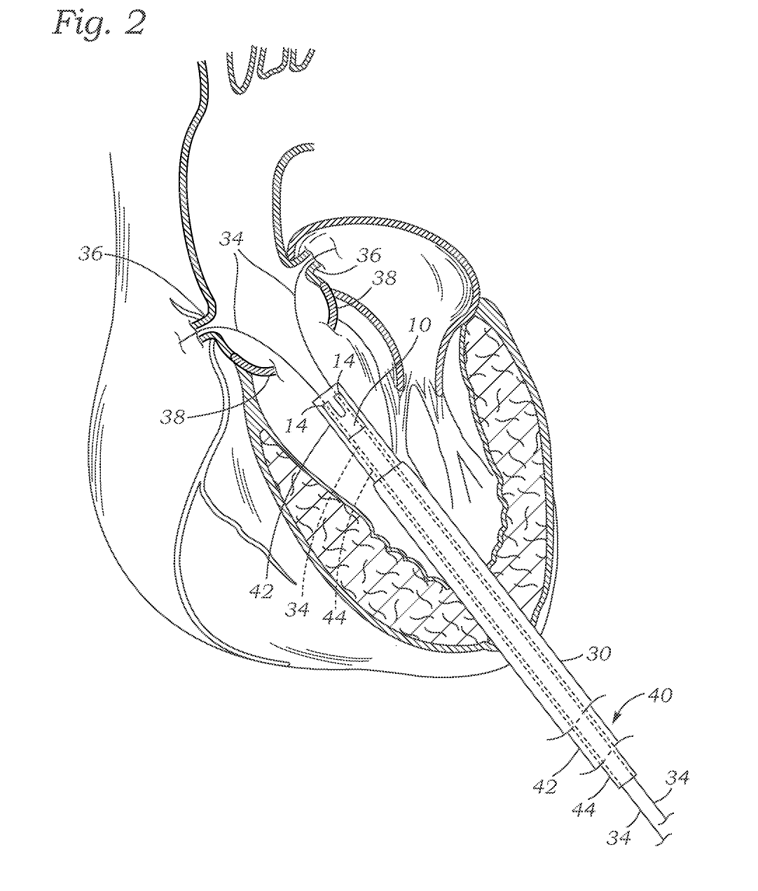

The graft 10 can be crimped (i.e., radially compressed) and loaded into a sheath 42 of a delivery apparatus 40 (FIG. 2) for introduction into the heart. Before or after the step of crimping and loading the graft into the sheath 42, the graft 10 is slid over the proximal ends of the guidewires 34 such that each guidewire extends through a side branch 14 and the lumen of the main body 12 of the graft. Thus, when the graft is loaded in the sheath 42 and ready for delivery into the heart, each guidewire 34 extends, in a proximal direction extending from the heart toward the surgeon, outwardly from a coronary artery 36, through the introducer sheath 30 and through the graft 10 and the delivery apparatus 40.

As depicted in FIG. 2, the delivery apparatus 40 (which contains the graft 10 in the sheath 42) can then be inserted through the introducer sheath 30 and along the guidewires 34 until the sheath 42 extends distally past the distal end of the introducer sheath 30. When the distal end of the sheath 42 is near the aortic root (e.g., just below or above the native leaflets 38), the graft 10 can be deployed from the sheath 42. To assist in deploying the graft 10, the delivery apparatus 40 can include a pusher mechanism or inner shaft 44, which can be used to push the graft distally through the distal opening of the sheath 42. Alternatively, the sheath 42 can be retracted relative to the graft 10 to effect deployment of the graft, in which case the inner shaft 44 can be used to hold the graft in place relative to the sheath 42 as the sheath 42 is retracted. After or as the graft 10 is being deployed from the sheath, the side branches 14 of the graft are directed into the coronary arteries 36 via the guidewires 34. The inner shaft 44 can be used to push the graft 10 along the guidewires 34 until the side branches 14 extend into the coronary arteries. FIG. 3 shows the graft 10 in its fully deployed position with the side branches 14 extending into the coronary arteries 36.

In some embodiments, the inner shaft 44 can form a releasable connection with the graft 10, which can allow a user to move the graft axially or rotationally by push/pull movements or rotational movements of the inner shaft 44 in order to achieve proper positioning of the graft with the side branches extending into the coronary arteries. When the graft is positioned at its final implantation position, the connection between the graft and the delivery apparatus can be released to permit removal of the delivery apparatus from the patient's body. Details of various releasable connections that can be incorporated in the present invention are disclosed in U.S. Patent Application Publication Nos. 2010/0049313 and 2012/0239142, which are incorporated herein by reference.

As noted above, the graft 10 has prosthetic leaflets 20 to help regulate the flow of blood from the left ventricle to the aorta. In the illustrated embodiment, the graft 10 is shown as being implanted in the aortic root just above the native leaflets 38. Thus, in this case, the leaflets 20 of the graft do not replace the native leaflets 38, which can continue to function. In the case of a patient with aortic insufficiency, the prosthetic leaflets 20 can prevent or minimize regurgitation through the native aortic valve. In another embodiment, the graft 10 can be implanted within the aortic annulus such that the graft is expanded against the native leaflets 38, in which case the prosthetic leaflets 20 completely replace the function of the native leaflets 38.

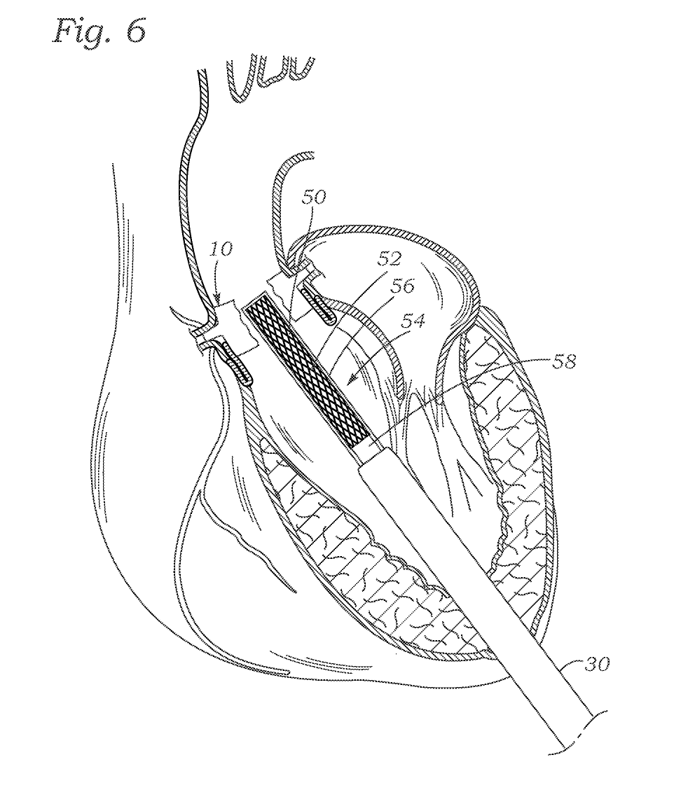

Referring now to FIG. 6, after implantation of the graft 10, a second graft 50 can be implanted downstream of the first graft 10 to reinforce a section of the ascending aorta, such as to treat dilation of the ascending aorta or an aneurism in that section of the ascending aorta. The graft 50, like graft 10, can be radially compressible and expandable for delivery into the body via catheterization. The graft 50 can be self-expandable or plastically-expandable. In the illustrated embodiment, the graft 50 comprises a frame 52 made of a self-expandable material (e.g., nitinol). The graft 50 also can include a blood-impermeable cover or liner, such as made of a synthetic fabric or natural tissue, supported by the frame 52.

FIG. 6 shows the graft 50 constrained in a radially compressed state within the sheath 56 of a delivery apparatus 54. The delivery apparatus 54 can further include an inner shaft or pusher member 58 to assist in deploying the graft 50 from the sheath 56. As shown, the delivery apparatus 54 can be inserted through the introducer sheath 30 to access the aorta. The delivery apparatus 54 can be advanced until the sheath 56 is located at the desired implantation location within the aorta, at which point the graft 50 can be deployed by retracting the sheath 56 relative to the inner shaft 58 and/or advancing the inner shaft 58 distally relative to the sheath 56. Positioning and deployment of the graft 50 can be aided by the use of techniques including fluoroscopy and/or ultrasound.

As shown in FIG. 7, the graft 50 can be implanted relative to the graft 10 such that an inflow end portion of the graft overlaps and engages an outflow end portion of the graft 10. In other embodiments, the graft 50 can be deployed immediately downstream of the graft 10 such that the two grafts are positioned end-to-end in an abutting relationship without any overlap or the graft 50 can be axially spaced downstream of the graft 10. The overall length of the graft 50 can vary depending on the particular condition of the patient. In the illustrated example, the graft 50 extends from the outflow of the aortic root to a location upstream of the branch arteries extending from the aortic arch (e.g., the brachiocephalic, left common carotid, and left subclavian arteries). In some embodiments, the graft 50 can extend into the aortic arch to a location downstream of one or more of the branch arteries, although the distal portion of the graft desirably is provided without a blood-impermeable cover or liner or selected portions are provided without a blood-impermeable cover or liner to permit blood flow into the branch arteries.

The graft 50 can have various shapes and/or configurations and can be delivered as multiple components. In one implementation, for example, a relatively long first stent can be deployed within the ascending aorta and/or the aortic arch, and a second stent having a blood-impermeable cover or liner (i.e., a stented graft) can be deployed within the first stent. In another implantation, the graft 50 can be replaced with any stented medical device that comprises an expandable stent and a structure configured to promote the flow of blood away from the dilated portion of the aorta. In this regard, the medical device can be referred to as a “deflector” in that it prevents or minimizes the flow of blood against selected portion(s) of the aorta. The deflector can have various shapes and/or configurations to address anatomical variations in size and positioning of the aneurism(s). For example, in one implantation, the deflector can comprise an expandable stent that supports a material that can extends into and fill an aneurism. The material can be an inflatable balloon, or an open or closed cell foam. Various embodiments of deflectors that can be incorporated in the present invention are disclosed in U.S. Patent Application Publication No. 2012/0310328, which is incorporated herein by reference.

After deployment of the graft or deflector 50, a prosthetic valve can be deployed in the sinus graft 10. The graft 10 can be used to support a wide variety of prosthetic valves delivered through a variety of mechanisms (e.g., self-expanding prosthetic valves, balloon-expandable prosthetic valves, and the like). For example, without limitation, any of the prosthetic valves disclosed in U.S. Pat. No. 6,730,118, U.S. Pat. No. 7,993,394, U.S. Pat. No. 8,652,202, U.S. Patent Application Publication No. 2012/0123529 and U.S. Patent Application Publication No. 2012/0239142, all of which prior patents and publications are incorporated herein by reference.

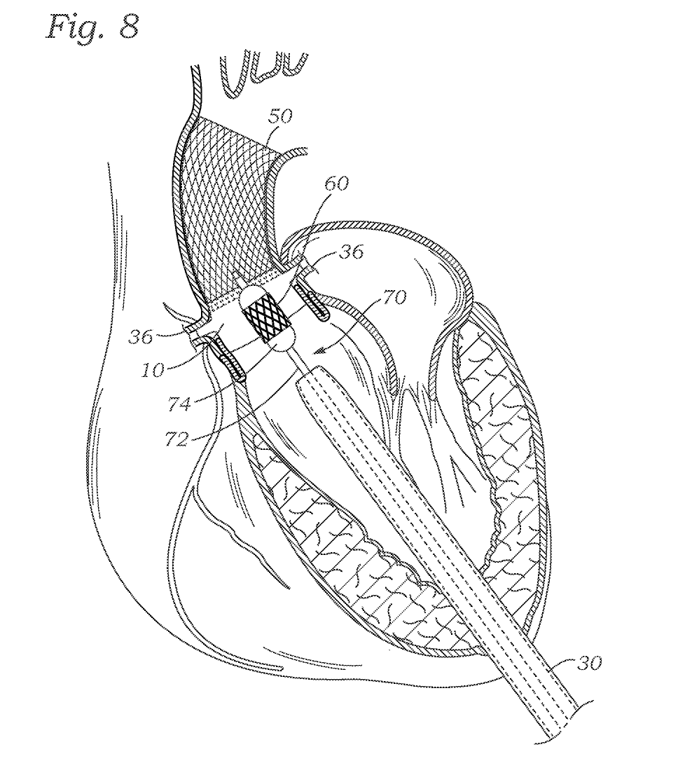

Referring then to FIG. 8, there is shown a prosthetic valve 60 being delivered to the sinus graft 10 using a delivery apparatus 70. The delivery apparatus 70 can comprise an elongated catheter or shaft 72 and an inflatable balloon 74 mounted on the distal end portion of the shaft 72. The prosthetic valve 60 can be crimped onto the balloon 74, as known in the art. The prosthetic valve 60 in the illustrated embodiment comprises a plastically-expandable frame or stent (e.g., made of stainless steel or a cobalt chromium alloy) supporting a plurality of prosthetic leaflets. As shown, the delivery apparatus 70 can be inserted into the left ventricle via the introducer sheath 30 and advanced distally until the prosthetic valve 60 is positioned at least partially within the sinus graft 10. Desirably, the outflow end of the prosthetic valve is positioned just below the coronary arteries 36 so as not to obstruct the flow of blood into the coronary arteries following deployment of the prosthetic valve.

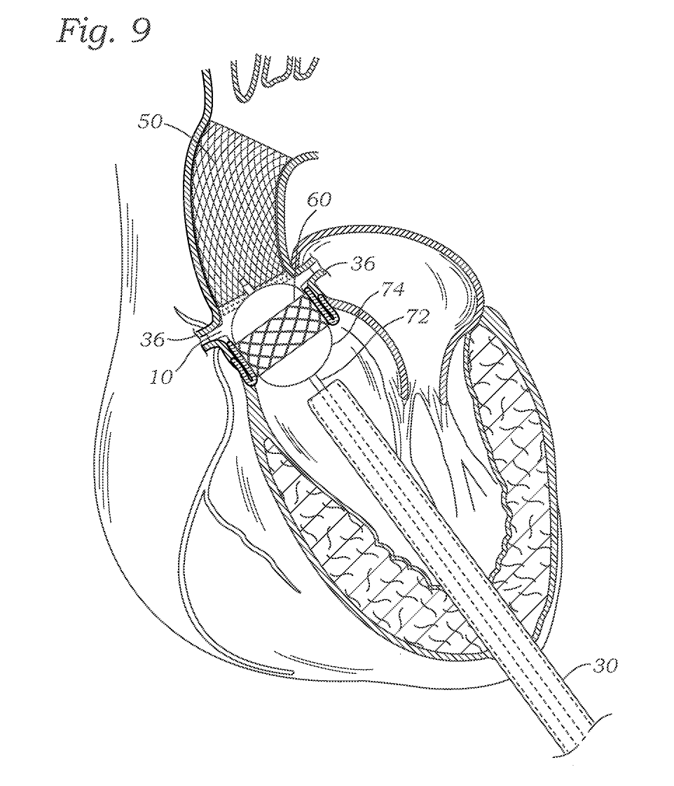

Once positioned at the desired implantation location, the balloon 74 can be inflated to expand the prosthetic valve against the inside surface of the graft 10, as depicted in FIG. 9. If the graft 10 is provided with prosthetic leaflets 20, the prosthetic valve 60 can be expanded against the prosthetic leaflets 20, thereby pushing the leaflets 20 against the inner surface of the frame 16. After expanding the prosthetic valve 60, the balloon 74 can be deflated and the delivery apparatus 70 can be removed from the heart, leaving the prosthetic valve 60 implanted within the sinus graft 10, as depicted in FIG. 10.

The lower portion of the sinus graft 10 is sufficiently rigid to support the prosthetic valve 60 and avoid further radial expansion upon expansion of the prosthetic valve 60 against the inner surface of sinus graft. Advantageously, the sinus graft 10 provides a suitable anchor or base for implanting prosthetic valve within or adjacent a dilated and/or non-calcified aortic annulus that otherwise might not reliably support a prosthetic valve, and in particular a plastically expandable prosthetic valve, which typically is not suitable for treating a dilated and/or non-calcified aortic. Depending on the size of the prosthetic valve 60, the prosthetic valve may extend downwardly into aortic annulus or the slightly into the left ventricle. In other implementations, the prosthetic valve 60 is positioned entirely within the aortic root downstream of the native leaflets 38.

In an alternative embodiment, the method of treatment need not include implanting a graft or deflector (e.g., a graft 50) in the ascending aorta. Thus, a prosthetic valve 60 can be implanted in the sinus graft 10 without an intervening step. In another embodiment, a graft or deflector (e.g., a graft 50) can be implanted in the ascending aorta after implanting the prosthetic valve 60 in the sinus graft 10.

FIG. 11 shows a sinus graft 100 according to another embodiment. The sinus graft 100 is similar to sinus graft 10, but instead of side branches 14, the sinus graft 100 has two apertures or openings 102 (one of which is shown in FIG. 11) extending through the frame and cover of the graft in place of the side branches 14. When implanted, the openings are aligned with the coronary arteries 36. The sinus graft 100, like graft 10, can have one or more prosthetic leaflets 20 (not shown in FIG. 11).

In some embodiments, the graft 100 can be manufactured without any openings 102. Prior to implantation, imaging techniques (CT scanning, ultrasound, etc.) can be used to identify the positions of the coronary ostia, and the surgeon can cut openings 102 in the cover of the graft at locations corresponding to the coronary ostia when the graft is implanted.

FIG. 12 shows another embodiment comprising sinus graft 100 and two separate side stents or branch conduits 104 that are delivered separately to the sinus graft. Each branch conduit 104 is configured to extend through an opening 102 in the graft 100 and into a coronary artery 36 to help anchor the graft in place. Each branch conduit 104 can comprise a radially compressible and expandable stent or frame, which can further include a blood-impermeable cover or liner supported by the frame. Each conduit 104 can include a generally cylindrical main body 106 and an enlarged flange 108 at one end of the main body. The frame of each conduit can be made of a self-expanding material (e.g., nitinol) or a plastically-expandable material (e.g., stainless steel or a cobalt chromium alloy).

Referring to FIG. 13, the sinus graft 100 is first deployed within the aortic root, using the guidewires 34 to align the openings 102 with the coronary arteries 36. Thereafter, the side stents 104 can be delivered along the guidewires 34, and advanced through the openings 102 into the coronary arteries 36. Once the main body 106 of a stent is advanced into a coronary artery 36, the stent can be expanded against the inner wall of the coronary artery. When the stent 104 is expanded, the flange 108 has a diameter larger than the opening 102 so as to retain the stent 104 relative to the graft 100. The stent 104 on the left hand side of FIG. 13 is shown fully advanced through the corresponding opening 102 and expanded against the inner wall of the coronary artery 36. The stent 104 of the right hand side of FIG. 13 is shown partially advanced through the corresponding opening 102 and prior to expansion of the stent.

FIG. 14 shows an embodiment comprising a sinus graft 200 that is similar to graft 10 in all respects expect that the former has a bulbous shaped main body 202 that generally corresponds to the shape of the aortic root, and thereby can have a central portion having a diameter that is larger than the diameters of the inflow and the outflow ends of the main body. The graft 200 can have side branches 204 adapted to extend into the coronary arteries or opening(s) in place of one or both of the side branches. The side branches 204 can be connected to the main body as shown or they can be separate components that are implanted after the main body is implanted (such as shown in FIGS. 12 and 13).

In another embodiment, a sinus graft (e.g., a graft 10, 100, or 200) can have prosthetic leaflets 20 that are sufficiently robust to last several months, years, or decades, in which case a separate prosthetic valve 60 would not be implanted in the sinus graft.

In certain embodiments, a sinus graft (e.g., a graft 10, 100, or 200) can be sized to have an inner diameter that is the same as or slightly greater than the expanded size of the prosthetic valve that is to be implanted within the graft. In some embodiments, a sinus graft can be manufactured in a plurality of different sizes, each corresponding to a size of the prosthetic valve that is to be implanted.

In another embodiment, a prosthetic device can comprise a single graft that has a first portion configured to be implanted within the aortic root and a second portion configured to be implanted within the ascending aorta. For example, the prosthetic device can comprise a first portion in the form of a sinus graft (e.g., sinus 10, 100, or 200) and a second portion in the form of graft 50. The first and second portions can be connected end-to-end or they can be interconnected to each other with longitudinally extending struts or tethers or sutures. A prosthetic device having such first and second portions can be mounted on the same delivery apparatus and delivered together to the aortic root and the ascending aorta, rather than in separate delivery steps.

In the illustrated embodiment, the guidewires 34, the graft 10, the graft 50, and the prosthetic valve 60 are delivered through a surgical opening in the wall of the left ventricle. However, other procedures can be utilized to deliver these components. In one implementation, one or more of these components can be delivered transfemorally in a retrograde approach through a femoral artery and the aorta. In another implementation, one or more of these components can be delivered transaortically through a surgical incision made in the ascending or descending aorta. In another implementation, one component can be delivered transfemorally, transaortically, or transventricularly, while another one of these components can be delivered by another one of these delivery approaches.

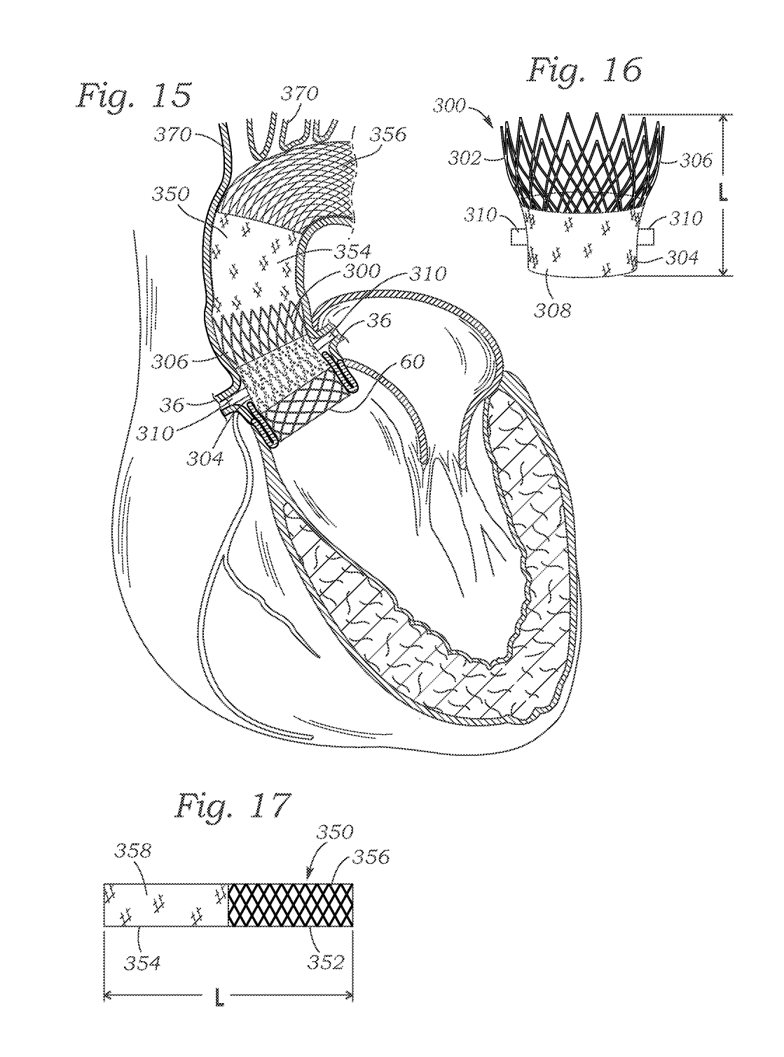

FIG. 15 shows another embodiment of a prosthetic assembly comprising a prosthetic valve 60, a first, sinus graft 300 and a second graft 350 implanted in the aortic valve, the aortic root and the ascending aorta, respectively. The second graft 350 can be sized to extend partially into the aortic arch, as depicted in FIG. 15. The grafts 300, 350 and the prosthetic valve 60 can be implanted using any of the delivery techniques and devices described above. The grafts 300 and 350, like grafts 10 and 50, can be radially compressible and expandable for delivery into the body via catheterization. The grafts 300, 350 can be self-expandable or plastically-expandable.

As best shown in FIG. 16, the sinus graft 300 comprises a frame 302 and a generally cylindrical inflow portion 304 and a flared outflow portion 306 that has a larger diameter than the inflow portion 304. In the illustrated embodiment, the frame 302 is made of a self-expandable material (e.g., nitinol), but can be made of plastically-expandable materials (e.g., stainless steel) in alternative embodiments. The inflow portion 304 can have a blood-impermeable cover or liner 308, such as made of a synthetic fabric or natural tissue, supported on the outside of the frame 302 (as shown in FIG. 16) and/or on the inside of the frame. The outflow portion 306 can be without a cover or liner on the outside or inside of the frame.

The outflow portion 306 desirably is without a cover or liner to permit blood flow through the outflow portion upon initial placement and to provide a greater retention force against the adjacent tissue of the aorta. Eliminating the cover on the outflow portion 306 also helps minimize the delivery profile of the sinus graft in its radially collapsed state and facilitates delivery of the sinus graft to its target implantation location.

The inflow portion 304 can also have side branches 310 adapted to extend into the coronary arteries or opening(s) in place of one or both of the side branches. Each of the side branches 310 can comprise an expandable annular stent or frame extending substantially perpendicularly from the frame 302. The frames of the side branches 310 optionally can be covered by the material forming the cover 308 as shown in FIG. 16. In some embodiments, the frames of the side branches 310 are covered by the cover 308 except for the distal end portions of the frames (the distal end portions being the end portions opposite the end portions connected to the inflow portion 304) to provide increased anchoring of the side branches in the coronary arteries.

In particular embodiments, the inflow portion 304 has an outer diameter in the expanded state of about 28 mm and the outflow portion 306 has an outer diameter in the expanded state of about 55 mm to about 70 mm. The sinus graft 300 can have a length or height L (FIG. 16) (from the inflow end to the outflow end) in the expanded state of about 30 mm to about 80 mm in some embodiments, about 30 mm to about 60 mm in some embodiments, and about 30 mm to about 50 mm in some embodiments. The outflow portion 306 can extend at least about 30 mm along the inner wall of the ascending aorta.

As best shown in FIG. 17, the second graft 350 comprises a generally tubular or cylindrical frame 352 and has an inflow portion 354 and an outflow portion 356. In the illustrated embodiment, the frame 352 is made of a self-expandable material (e.g., nitinol), but can be made of plastically-expandable materials (e.g., stainless steel) in alternative embodiments. The inflow portion 354 can have a blood-impermeable cover or liner 358, such as made of a synthetic fabric or natural tissue, supported on the outside of the frame 352 (as shown in FIG. 17) and/or on the inside of the frame.

The second graft 350 has an overall length or height L (FIG. 17) in the expanded state, for example, of at least about 30 mm to about 100 mm in some embodiments, about 30 mm to about 80 mm in some embodiments, and about 30 mm to about 60 mm in some embodiments. The cover 358 desirably covers about half of the length of the graft 350.

The sinus graft 300 can be implanted first such that the side branches 310 extend into the coronary arteries 36. The flared outflow portion 306 can be placed in a dilated portion of the ascending aorta. Following implantation of the sinus graft 300, the second graft 350 can be implanted such that the inflow portion 354 is placed in the outflow portion 306 of the sinus graft 300 in the ascending aorta and the outflow portion 356 extends partially into aortic arch. The end of the inflow portion 354, for example, can be placed at the level of the outflow end of the cover 308 of the sinus graft, or just below the outflow end of the cover 308 such that the cover 308 overlaps the adjacent end portion of the second graft 350. The outflow portion 356 of the second graft 350 can extend past one or more branch arteries 370 as shown. Blood flowing into the aortic arch can flow outwardly through the openings in the outflow portion 356 into the branch arteries 370. The cover 358 extending over the inflow portion 354 of the second graft creates a seal with the inner surface of the outflow portion 306 of the sinus graft.

Before or after implanting the second graft 350, the prosthetic valve 60 can be implanted such that at least an outflow portion of the prosthetic valve 60 is deployed within the inflow portion 304 of the sinus graft 300. For example, the outflow end of the prosthetic valve 60 can be positioned within the sinus graft 300 just below the side branches 310. The prosthetic valve 60 can have a blood-impermeable liner or cover that covers a part of or the entirety of the outer surface of the frame of the prosthetic valve and/or the inner surface of the frame of the prosthetic valve. Thus, when all three components are implanted as shown in FIG. 15, a continuous covered conduit is formed that extends from the aortic valve to a location immediately upstream of the first branch artery.

In certain embodiments, the sinus graft 300 can have prosthetic leaflets 20 that are sufficiently robust to last several months, years, or decades, in which case a separate prosthetic valve 60 would not be implanted in the sinus graft.

In some embodiments, additional coronary stents can be implanted within the side branches 310 to help maintain the patency of the side branches.

In some embodiments, the inflow portion 304 of the sinus graft has axially extending projections or formations that are configured to be implanted within the sinuses behind the native leaflets of the aortic valve, such as disclosed in the above-mentioned U.S. Publication No. 2012/0310328. In such embodiments, the projections or formations are implanted radially outside of the native leaflets and the prosthetic valve 60 is implanted radially inside of the native leaflets such that the native leaflets are captured and compressed between the prosthetic valve and the projections or formations of the sinus graft. The projections or formations positioned radially outside of the native leaflets help anchor the prosthetic valve 60 in place, especially in a dilated aortic annulus having little or no calcification.

### GENERAL CONSIDERATIONS

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods. As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A”, “B”, “C”, “A and B”, “A and C”, “B and C”, or “A, B, and C”.

As used herein, the term “coupled” generally means physically coupled or linked and does not exclude the presence of intermediate elements between the coupled items absent specific contrary language.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

I claim:

1. A method comprising:
introducing a guidewire into a patient's body;
advancing the guidewire until a distal end portion of the guidewire extends into the aortic root and into one of the coronary arteries;
advancing a prosthetic device along the guidewire into the aortic root;
aligning a side opening of the prosthetic device with the coronary artery into which the guidewire extends; and
radially expanding the prosthetic device within the aortic root.

2. The method of claim 1, further comprising implanting a prosthetic valve within the prosthetic device.

3. The method of claim 2, wherein implanting the prosthetic valve within the prosthetic device comprises introducing the prosthetic valve into the patient's body on a catheter and radially expanding the prosthetic valve within the prosthetic device.

4. The method of claim 1, wherein the prosthetic device comprises prosthetic valve leaflets.

5. The method of claim 1, further comprising implanting a stented graft in the ascending aorta.

6. The method of claim 5, wherein an inflow end portion of the stented graft overlaps an outflow end portion of the prosthetic device.

7. The method of claim 1, wherein:
the act of introducing a guidewire into a patient's body comprises introducing first and second guidewires into the patient's body;
the act of advancing the guidewire comprises advancing the first and second guidewires until distal end portions of the guidewires extend into the aortic root and each distal end portion extends into one of the coronary arteries;
the act of advancing a prosthetic device comprises advancing the prosthetic device along the first and second guidewires into the aortic root; and
the act of aligning a side opening of the prosthetic device comprises aligning first and second side openings of the prosthetic device with the coronary arteries.

8. The method of claim 7, wherein prior to the act of advancing the prosthetic device along the first and second guidewires, placing the prosthetic device on a delivery apparatus and inserting the proximal ends of the first and second guidewires through the first and second side openings of the prosthetic device.

9. The method of claim 1, further comprising implanting a branch conduit in the coronary artery into which the guidewire extends, one end of the branch conduit being in communication with the side opening of the prosthetic device to allow blood to flow outwardly through the branch conduit into the coronary artery.

10. The method of claim 9, wherein the prosthetic device comprises an annular main body and the branch conduit, which is connected to the main body, wherein the main body and the branch conduit are delivered to the aortic root at the same time.

11. The method of claim 9, wherein the prosthetic device comprises an annular main body that is radially expanded in the aortic root and the branch conduit is separate from the main body and is inserted into the patient and implanted after the main body is implanted.

12. The method of claim 1, wherein an inflow end of the prosthetic device is implanted above the native aortic valve leaflets.

13. The method of claim 1, wherein radially expanding the prosthetic device causes the prosthetic device to engage the inner wall of the aortic root.

14. An implantable prosthetic device configured for implantation in the aortic root of a patient, the prosthetic device comprising:
an annular body configured to be radially compressed to a delivery state for insertion into the patient and expandable to an expanded state against the inner wall of the aortic root; and
first and second openings in the annular body and configured to allow blood to flow outwardly through the openings and into the coronary arteries when the annular body is in an expanded state engaging the inner wall of the aortic root.

15. The prosthetic device of claim 14, further comprising at least one branch conduit extending from one of the first and second openings and configured to be implanted within one of the coronary arteries.

16. The prosthetic device of claim 15, wherein the at least one branch conduit comprises first and second branch conduits extending from the first and second side openings, respectively, and configured to be implanted within the coronary arteries.

17. The prosthetic device of claim 15, wherein the at least one branch conduit is separate from the annular body.

18. The prosthetic device of claim 14, wherein the annular body comprises a radially compressible and expandable metal frame and a blood-impermeable liner supported by the frame.

19. The prosthetic device of claim 14, further comprising prosthetic valve leaflets supported within the annular body.

20. The prosthetic device of claim 15, wherein the branch conduit is radially compressible for delivery into the patient and radially expandable to an expanded state to engage an inner wall of the coronary artery.

21. A medical device assembly comprising:
first and second guidewires;
an elongated delivery apparatus having a distal end portion; and
an implantable prosthetic device configured to be implanted within the aortic root of a patient's body, the prosthetic device being mounted in a radially compressed state on the distal end portion of the delivery apparatus, the prosthetic device comprising an annular body and first and second openings in the annular body and configured to allow blood to flow outwardly through the openings and into the coronary arteries when the annular body is in an expanded state engaging the inner wall of the aortic root;
wherein the first and second guidewires extend into and through the first and second openings, respectively, and through the annular body.
